(19) United States
(12) Patent Application Publication
(10) Pub. No.: US 2005/0059646 A1
(43) Pub. Date: Mar. 17, 2005

(54) Use of 4-androstene-3,6,17-trione to elevate testosterone levels and the testosterone/estrogen ratio in males

(75) Inventor: Patrick Arnold, Champaign, IL (US)

Correspondence Address: MICHAEL BERNS;MALONEY, PARKINSON AND BERNS, 135 W MAIN STREET, URBANA, IL, 61801, US

(73) Assignee: Proviant Technology Inc., Champaign, IL (US)

(21) Appl. No.: 10/662,594

(22) Filed: Sep. 15, 2003

(51) Int. Cl. A61K 31/57

(52) U.S. Cl. 514/177

(57) ABSTRACT

The present invention involves the use of 4-androstene-3,6,17-trione to stimulate endogenous testosterone production in males, while leaving estrogen levels relatively unaffected. It also involves the use of 4-androstene-3,6,17-trione to increase the testosterone/estrogen (T/E) ratio in males.

### FIELD OF THE INVENTION

The present invention involves the use of 4-androstene-3,6,17-trione to stimulate endogenous testosterone production in males, while leaving estrogen levels relatively unaffected. It also involves the use of 4-androstene-3,6,17-trione to increase the testosterone/estrogen (T/E) ratio in males.

### BACKGROUND OF THE INVENTION

Testosterone is the hormone responsible for secondary sexual characteristics in males. Normal levels of testosterone are necessary for the full expression of the physical, psychological, and sexual characteristics of mature manhood. Estrogen is the hormone responsible for the secondary sexual characteristics in females. Males too produce estrogen, and its presence in precise amounts is necessary for the activity of testosterone to be fully generated.

### SUMMARY OF THE INVENTION

It is an object of the invention to provide a method of increasing testosterone levels while preventing increases in estrogen levels. Administration of 4-androstene-3,6,17-trione has been found to be effective and successful.

### DETAILED DESCRIPTION OF THE PREFERRED EMBODIMENTS

While absolute levels of testosterone and estrogen are of great importance to the male, the ratio of testosterone to estrogen (T/E ratio) should also be within a certain range for a male to exhibit maximal endocrinological health. Specifically, it is the presence of an abnormally low T/E ratio that is most problematic. If this ratio is too low then estrogen related disorders like gynecomastia and excessive bodyfat deposition can develop. Sex drive may suffer. Estrogen also increases the production of Sex Hormone Binding Globulin (SHBG), which then further decreases the biologically active free testosterone available in the system. Additionally, a low testosterone/estrogen ratio has been associated with the increase risk of developing benign prostate hypertrophy (BPH).

It is important to note here however that estrogen is still an important hormone in men, and normal levels of estrogen are necessary for optimal bone density, cognitive function, cardiovascular health, and sexual function.

As men increase in age, a gradual decrease in testosterone production is often seen. A decrease in testosterone production can be responsible for many age related disorders such as deceased strength and muscle mass, decreased cognitive function, and decreased libido.

The decrease in testosterone production in older males often is not accompanied by a concomitant decrease in estrogen production. Consequently, an undesirable T/E ratio is then established. It is once again important to note that this decrease is not representative of an unhealthy increase in estrogens, but rather due to a decrease in testosterone levels towards to the low normal or subphysiological range.

The usual treatment for testosterone deficiency is the administration of exogenous androgens. These are given usually by injection (i.e. testosterone cypionate), transdermal administration (i.e. Andro-Gel®) or by the administration of synthetic orally active androgens (i.e. methyltestosterone). The orally active androgens however have significant liver toxicity so they have gone out of favor. Injections are less toxic but they are inconvenient and do not give steady blood levels of hormone. Transdermal androgens are the most convenient and pharmacokinetically favorable however even they are not without drawbacks. All exogenous androgens, including transdermals, lead to shutdown of the hypothalamic pituitary testicular axis (HPTA) and consequently induce testicular atrophy. Furthermore, exogenous androgens do not necessarily ameliorate the abnormally high T/E ratio seen in many older hypogonadal males. The exogenous androgens can still over-aromatize to estrogens and so even though androgen levels are back to normal, the T/E ratio may remain relatively unchanged.

It is known that the HPTA is regulated by both estrogens and androgens in males. The more powerful regulator however is estrogen. In males, circulating testosterone is extensively metabolized into estradiol at the hypothalamus, and this estradiol then is available to immediately bind to hypothalamic estrogen receptors. The end results is a decrease in the secretion of gonadotropin releasing hormone (GnRH).

GnRH is a hormone that is responsible for signaling the pituitary gland to release gonadotropins—specifically LH and FSH. These hormones then are released into the bloodstream where they travel to the testes and stimulate the production of testosterone and the synthesis of sperm.

It is also known that administration of anti-estrogen drugs to males causes a pronounced up-regulation of testosterone production by interfering with the normal estrogen mediated negative feedback system that starts at the hypothalamus.

There are two kinds of anti-estrogen drugs. The first class is estrogen receptor antagonists (ERA's). Examples of these are tamoxifen and clomiphene. These drugs bind to the estrogen receptor but do not activate the estrogen responsive genes like normal estrogens do. They compete with the estrogen receptor and block out the endogenous active estrogens. These drugs however typically have a certain degree of pro-estrogenic activity and therefore can act like real estrogens at certain tissues. They therefore are not purely anti-estrogenic.

Furthermore, ERA's lead to an increase in the levels of estrogens in the blood due to their stimulatory effect on the production of androgens—which are estrogen precursors. As a result, there can be a significant “estrogenic rebound” when the ERA's are discontinued.

The other kind of anti-estrogens are aromatase inhibitors. Examples of these are testolactone and anastrazole. These compounds work by blocking and inactivating the aromatase enzyme. The aromatase enzyme is responsible for the formation of estrogens from androgenic precursors. In men, the two major precursors for estrogen biosynthesis are testosterone and androstenedione. Aromatase inhibitors therefore prevent the actual formation of estrogen in the body, as opposed to receptor antagonists which merely block the activity of circulating estrogens. Since aromatase inhibitors do not lead to any sort of incidental pro-estrogenic activity like the receptor antagonists do, they are the cleaner anti-estrogens and therefore more preferred.

Also, with aromatase inhibitors there is no estrogenic rebound upon discontinuation, since estrogen levels are not elevated. There simply is a gradual return to the baseline homeostatic sex hormone levels that were present before therapy was commenced.

4-androstene-3,6,17-trione (a-trione) is a metabolite of androstenedione that has been shown to have aromatase inhibiting activity in-vitro. It has never been tested in humans however, and in the course of our research we decided to examine its effect on the male endocrinological profile.

What we discovered was that a-trione produced a very marked increase of testosterone while exhibiting only a small, barely significant decrease in estradiol. This indicated that an improvement in the T/E ratio was achieved, while estrogen levels were still maintained within the healthy normal range. This makes the use of a-trione superior to most other aromatase inhibitors as a means of improving the T/E ratio. Specifically, because a-trione does not adversely effect estrogen production while other aromatase inhibitors tend to suppress estrogens down to the subphysiological range.

### EXAMPLE

Six male subjects, aged 32-40 years of age, were prescreened via a medical doctor for endocrinological abnormalities. The subjects' physical characteristics were: height 177.38+/−8057 cm, weight 88.8+/−13.65 kg, body fat percentage 14.9+/−3.5%. Subjects ingested 300 mg a-trione bid for three weeks as part of an open label design. Resting AM blood draws were taken at 0, 1, 2, and 3 weeks of supplementation. Table 1 indicates the changes in endocrine markers over the time evaluated. Results were analyzed using a Repeated Measures-ANOVA design with paired samples t-tests as appropriate. The results indicate that a-trione can increase testosterone levels (+88%) substantially while affecting estrogen only slightly (−11%). Therefore, a-trione can raise the T/E ratio while leaving estradiol in the safe physiological range.

| Endocrine Marker | Week 0 | Week 1 | Week 2 | Week 3 |
|---|---|---|---|---|
| Total Testosterone | 443.67 ± | 701.17 ± | 752.17 ± | 835.33 ± |
| (ng/dL) | 59.07 | 36.85 | 78.11 | 124.74 |
| Free Testosterone | 126.67 ± | 216.67 ± | 252.33 ± | 285.67 ± |
| (nmol/L) | 31.99 | 33.64 | 53.40 | 69.22 |
| Sex Hormone Binding | 21.83 ± | 20.5 ± | 19.5 ± | 18.83 ± |
| Globulin (SHBG) | 4.36 | 3.83 | 5.54 | 6.18 |
| (nmol/L) | | | | |
| Estradiol (pg/mL) | 16.5 ± | 16.5 ± | 14.8 ± | 14.67 ± |
| | 1.38 | 2.93 | 2.93 | 2.94 |
| Dihydrotestosterone | 33.33 ± | 39.83 ± | 45.83 ± | 47.5 ± |
| (ng/dL) | 3.56 | 7.88 | 14.72 | 8.17 |
| T/E Ratio | 269/1 | 425/1 | 508/1 | 569/1 |

The average optimal daily dose of a-trione is 600 mg a day. The effective dosage range however can extend from 100 mg to 1000 mg per day.

A-trione can be administered orally, by injection, transdermally, intranasally, sublingually, or by any other commonly accepted pharmaceutical route. For convenience purposes however the preferred mode of administration is oral.

Oral compositions may include tablets, capsules, dragees, liquid suspensions, or any other common oral formulation.

1) A method of increasing endogenous testosterone production in men through the administration of 4-ANDROSTENE-3,6,17-TRIONE

2) A method of increasing the testosterone/estrogen ratio in men through the administration of 4-ANDROSTENE-3,6,17-TRIONE

3) A method of increasing testosterone levels in men while leaving estrogen levels relatively unaffected through the administration of 4-ANDROSTENE-3,6,17-TRIONE.

4) The method according to claim 1 where the dose administered is between 100 mg to 1000 mg per day.

5) The method according to claim 2, wherein the dose administered is between 100 mg to 1000 mg per day.

6) The method according to claim 3, wherein the dose through the administration of 4-ANDROSTENE-3,6,17-TRIONE
